(19) United States
(12) Patent Application Publication
(10) Pub. No.: US 2016/0367484 A1
(43) Pub. Date: Dec. 22, 2016

(54) PROCESS FOR THE PRODUCTION OF AN ABUSE-PROOFED SOLID DOSAGE FORM

(71) Applicant: GRÜNENTHAL GMBH, Aachen (DE)

(72) Inventors: Elisabeth ARKENAU-MARIC, Koln (DE); Johanness BARTHOLOMÄUS, Aachen (DE); Heinrich KUGELMANN, Aachen (DE)

(73) Assignee: GRÜNENTHAL GMBH, Aachen (DE)

(21) Appl. No.: 15/252,764

(22) Filed: Aug. 31, 2016

Related U.S. Application Data
(63) Continuation of application No. 14/935,609, filed on Nov. 9, 2015.
Application No. 14/935,609 is a continuation of application No. 14/657,401, filed on Mar. 13, 2015, now abandoned.
Application No. 14/657,401 is a continuation of application No. 14/143,437, filed on Dec. 30, 2013, now abandoned.
Application No. 14/143,437 is a continuation of application No. 12/140,531, filed on Jun. 17, 2008, now abandoned.
Application No. 12/140,531 is a division of application No. 10/890,703, filed on Jul. 14, 2004, now abandoned.

(30) Foreign Application Priority Data
Apr. 22, 2004 (DE) 10 2004 020 220.6

(51) Int. Cl. A61K 9/20; A61K 31/485; A61K 9/00

(52) U.S. Cl. CPC A61K 9/2095; A61K 9/0053; A61K 31/485; A61K 9/2077

(57) ABSTRACT

The present invention relates to a process for the production of an abuse-proofed solid dosage form containing at least one active ingredient with potential for abuse and a binder with a breaking strength of ≧500 N, by exposing a mixture comprising the active ingredient and the binder to ultrasound and force.

This application is a continuation of U.S. patent application Ser. No. 14/935,609, filed Nov. 9, 2015, now pending, which is, in turn, a continuation of U.S. patent application Ser. No. 14/657,401, filed Mar. 13, 2015, now abandoned, which is, in turn, a continuation of U.S. patent application Ser. No. 14/143,437, filed on Dec. 30, 2013, now abandoned, which is, in turn, a continuation of U.S. patent application Ser. No. 12/140,531, filed on Jun. 17, 2008, now abandoned, which is, in turn, a division of U.S. patent application Ser. No. 10/890,703, filed on Jul. 14, 2004, now abandoned, which, in turn, claims priority of German Patent Application No. 10 2004 020 220.6, filed on Apr. 22, 2004, the entire contents of which patent applications are incorporated herein by reference.

The present invention relates to a process for the production of an abuse-proofed solid dosage form containing at least one active ingredient with potential for abuse and a binder with a breaking strength of ≧500 N, by exposing a mixture comprising the active ingredient and the binder to ultrasound and force.

Many pharmaceutical active ingredients, in addition to having excellent activity in their appropriate application, also have potential for abuse, i.e. they can be used by an abuser to bring about effects other than those intended.

Opiates, for example, which are highly active in combating severe to very severe pain, are frequently used by abusers to induce a state of narcosis or euphoria.

In order to make abuse possible, the corresponding dosage forms, such as tablets or capsules are comminuted, for example ground in a mortar, by the abuser, the active ingredient is extracted from the resultant powder using a preferably aqueous liquid and the resultant solution, optionally after being filtered through cotton wool or cellulose wadding, is administered parenterally, in particular intravenously. An additional phenomenon of this kind of administration, in comparison with abusive oral administration, is a further accelerated increase in active ingredient levels giving the abuser the desired effect, namely the “kick” or “rush”. This kick is also obtained if the powdered dosage form is administered nasally, i.e. is sniffed. Since delayed-release dosage forms containing active ingredients with potential for abuse do not give rise to the kick desired by the abuser when taken orally even in abusively high quantities, such dosage forms are also comminuted and extracted in order to be abused.

U.S. Pat. No. 4,070,494 proposed adding a swellable agent to the dosage form in order to prevent abuse. When water is added to extract the active ingredient, this agent swells and ensures that the filtrate separated from the gel contains only a small quantity of active ingredient.

The multilayer tablet disclosed in WO 95/20947 is based on a similar approach to preventing parenteral abuse, said tablet containing the active ingredient with abuse potential and at least one gel former, each in different layers.

WO 03/015531 A2 discloses another approach to preventing parenteral abuse. A dosage form containing an analgesic opioid and a dye as an aversive agent is described therein. The colour released by tampering with the dosage form is intended to discourage the abuser from using the dosage form which has been tampered with.

Another known option for complicating abuse involves adding antagonists to the active ingredients to the dosage form, for example naloxone or naltexone in the case of opiates, or compounds which cause a physiological defence response, such as for example ipecacuanha (ipecac) root.

Since, however, as in the past, it is in most cases necessary for the purposes of abuse to pulverise the dosage form, it was the object of the present invention to provide a process for the production of dosage forms for active ingredients with potential for abuse, which, when correctly administered, ensure the desired therapeutic action, but from which the active ingredients cannot be converted into a form suitable for abuse simply by pulverisation.

This object has been achieved by the provision of the process according to the invention for the production of an abuse-proofed solid dosage form containing at least one active ingredient with potential for abuse and at least one binder with a breaking strength of ≧500 N, by exposing a mixture comprising the active ingredient and the binder to ultrasound and force.

By means of the production process according to the invention using ultrasound, it is possible to provide a dosage form with a breaking strength of ≧500 N which is capable of considerably complicating or preventing pulverisation of the dosage form with conventional means and any subsequent abuse.

If comminution is inadequate, parenteral, in particular intravenous, administration cannot be performed safely or extraction of the active ingredient therefrom takes too long for the abuser or there is no “kick” when orally abused, as release is not instantaneous.

According to the invention, comminution is taken to mean pulverisation of the solid dosage form with conventional means which are available to an abuser, such as for example a pestle and mortar, a hammer, a mallet or other usual means for pulverisation by application of force.

The process according to the invention for the production of dosage forms is accordingly suitable for preventing parenteral, nasal and/or oral abuse of active ingredients with potential for abuse.

Active ingredients with potential for abuse, preferably pharmaceutical active ingredients with potential for abuse, are known to the person skilled in the art, as are the quantities thereof to be used, and may be protected against abuse as such, in the form of the corresponding derivatives thereof, in particular esters or amides, or in each case in the form of corresponding physiologically acceptable compounds, in particular in the form of the salts or solvates thereof, as racemates, enantiomers or stereoisomers by the process according to the invention.

The process according to the invention is in particular suitable for preventing the abuse of a pharmaceutical active ingredient, which is from the group comprising narcotic analgesics, opiates, opioids, tranquillisers, preferably benzodiazepines, barbiturates, stimulants and further narcotics.

The process according to the invention is very particularly preferably suitable for preventing the abuse of at least one opiate, opioid, tranquilliser or at least one other narcotic which is selected from the group comprising N-{1-[2-(4-ethyl-5-oxo-2-tetrazolin-1-yl)ethyl]-4-methoxymethyl-4-piperidyl}propionanilide (alfentanil), 5,5-diallylbarbituric acid (allobarbital), allylprodine, alphaprodine, 8-chloro-1-methyl-6-phenyl-4H-[1,2,4]triazolo[4,3-a][1,4]-benzodiazepine (alprazolam), 2-diethylaminopropiophenone (amfepramone), (±)-α-methylphenethylamine (amphetamine), 2-(α-methylphenethylamino)-2-phenylacetonitrile (amphetaminil), 5-ethyl-5-isopentylbarbituric acid (amobarbital), anileridine, apocodeine, 5,5-diethylbarbituric acid (barbital), benzylmorphine, bezitramide, 7-bromo-5-(2-pyridyl)-1H-1,4-benzodiazepin-2(3H)-one (bromazepam), 2-bromo-4-(2-chlorophenyl)-9-methyl-6H-thieno[3,2-f][1,2,4]triazolo[4,3-a][1,4]diazepine (brotizolam), 17-cyclopropylmethyl-4,5α-epoxy-7α[(S)-1-hydroxy-1,2,2-trimethyl-propyl]-6-methoxy-6,14-endo-ethanomorphinan-3-ol (buprenorphine), 5-butyl-5-ethylbarbituric acid (butobarbital), butorphanol, (7-chloro-1,3-dihydro-1-methyl-2-oxo-5-phenyl-2H-1,4-benzodiazepin-3-yl) dimethylcarbamate (camazepam), (1S,2S)-2-amino-1-phenyl-1-propanol (cathine/D-norpseudoephedrine), 7-chloro-N-methyl-5-phenyl-3H-1,4-benzodiazepin-2-ylamine 4-oxide (chlordiazepoxide), 7-1-methyl-5-phenyl-1H-1,5-benzodiazepine-2,4(3H,5H)-dione (clobazam), 5-(2-chlorophenyl)-7-nitro-1H-1,4-benzodiazepin-2(3H)-one (clonazepam), clonitazene, 7-chloro-2,3-dihydro-2-oxo-5-phenyl-1H-1,4-benzodiazepine-3-carboxylic acid (clorazepate), 5-(2-chlorophenyl)-7-ethyl-1-methyl-1H-thieno[2,3-e][1,4]diazepin-2(3H)-one (clotiazepam), 10-chloro-11b-(2-chlorophenyl)-2,3,7,11b-tetrahydrooxazolo[3,2-d][1,4]benzodiazepin-6(5H)-one (cloxazolam), (−)-methyl-[3β-benzoyloxy-2β(1αH,5αH)-tropane carboxylate] (cocaine), 4,5α-epoxy-3-methoxy-17-methyl-7-morphinen-6α-ol (codeine), 5-(1-cyclohexenyl)-5-ethylbarbituric acid (cyclobarbital), cyclorphan, cyprenorphine, 7-chloro-5-(2-chlorophenyl)-1H-1,4-benzodiazepin-2(3H)-one (delorazepam), desomorphine, dextromoramide, (+)-(1-benzyl-3-dimethylamino-2-methyl-1-phenylpropyl)propionate (dextropropoxyphene), dextromethorphan, dezocine, diampromide, diamorphone, 7-chloro-1-methyl-5-phenyl-1H-1,4-benzodiazepin-2(3H)-one (diazepam), 4,5α-epoxy-3-methoxy-17-methyl-6α-morphinanol (dihydrocodeine), 4,5α-epoxy-17-methyl-3,6a-morphinandiol (dihydromorphine), dimenoxadol, dimephetamol, dimethylthiambutene, dioxaphetyl butyrate, dipipanone, (6aR,10aR)-6,6,9-trimethyl-3-pentyl-6a,7,8,10a-tetrahydro-6H-benzo[c]chromen-1-ol (dronabinol), eptazocine, 8-chloro-6-phenyl-4H-[1,2,4]triazolo[4,3-(a)][1,4]benzodiazepine (estazolam), ethoheptazine, ethylmethylthiambutene, ethyl [7-chloro-5-(2-fluorophenyl)-2,3-dihydro-2-oxo-1H-1,4-benzodiazepine-3-carboxylate] (ethyl loflazepate), 4,5α-epoxy-3-ethoxy-17-methyl-7-morphinen-6α-ol (ethylmorphine), etonitazene, 4,5α-epoxy-7α-(1-hydroxy-1-methylbutyl)-6-methoxy-17-methyl-6,14-endo-etheno-morphinan-3-ol (etorphine), N-ethyl-3-phenyl-8,9,10-trinorbornan-2-ylamine (fencamfamine), 7-[2-(α-methylphenethylamino)ethyl]-theophylline) (fenethylline), 3-(α-methylphenethylamino)propionitrile (fenproporex), N-(1-phenethyl-4-piperidyl)propionanilide (fentanyl), 7-chloro-5-(2-fluorophenyl)-1-methyl-1H-1,4-benzodiazepin-2(3H)-one (fludiazepam), 5-(2-fluorophenyl)-1-methyl-7-nitro-1H-1,4-benzodiazepin-2(3H)-one (flunitrazepam), 7-chloro-1-(2-diethylaminoethyl)-5-(2-fluorophenyl)-1H-1,4-benzodiazepin-2(3H)-one (flurazepam), 7-chloro-5-phenyl-1-(2,2,2-trifluoroethyl)-1H-1,4-benzodiazepin-2(3H)-one (halazepam), 10-bromo-11b-(2-fluorophenyl)-2,3,7,11b-tetrahydro[1,3]oxazolyl[3,2-d][1,4]benzodiazepin-6(5H)-one (haloxazolam), heroin, 4,5α-epoxy-3-methoxy-17-methyl-6-morphinanone (hydrocodone), 4,5α-epoxy-3-hydroxy-17-methyl-6-morphinanone (hydromorphone), hydroxypethidine, isomethadone, hydroxymethylmorphinan, 11-chloro-8,12b-dihydro-2,8-dimethyl-12b-phenyl-4H-[1,3]oxazino[3,2-d][1,4]benzodiazepine-4,7(6H)-dione (ketazolam), 1-[4-(3-hydroxyphenyl)-1-methyl-4-piperidyl]-1-propanone (ketobemidone), (3S,6S)-6-dimethylamino-4,4-diphenylheptan-3-yl acetate (levacetylmethadol (LAAM)), (−)-6-dimethylamino-4,4-diphenol-3-heptanone (levomethadone), (−)-17-methyl-3-morphinanol (levorphanol), levophenacylmorphane, levoxemacin, lofentanil, 6-(2-chlorophenyl)-2-(4-methyl-1-piperazinylmethylene)-8-nitro-2H-imidazo[1,2-a][1,4]-benzodiazepin-1(4H)-one (loprazolam), 7-chloro-5-(2-chlorophenyl)-3-hydroxy-1H-1,4-benzodiazepin-2(3H)-one (lorazepam), 7-chloro-5-(2-chlorophenyl)-3-hydroxy-1-methyl-1H-1,4-benzodiazepin-2(3H)-one (lormetazepam), 5-(4-chlorophenyl)-2,5-dihydro-3H-imidazo[2,1-a]isoindol-5-ol (mazindol), 7-chloro-2,3-dihydro-1-methyl-5-phenyl-1H-1,4-benzodiazepine (medazepam), N-(3-chloropropyl)-α-methylphenethylamine (mefenorex), meperidine, 2-methyl-2-propyltrimethylene dicarbamate (meprobamate), meptazinol, metazocine, methylmorphine, N,α-dimethylphenethylamine (metamphetamine), (±)-6-dimethylamino-4,4-diphenol-3-heptanone (methadone), 2-methyl-3-o-tolyl-4(3H)-quinazolinone (methaqualone), methyl [2-phenyl-2-(2-piperidyl)acetate] (methylphenidate), 5-ethyl-1-methyl-5-phenylbarbituric acid (methylphenobarbital), 3,3-diethyl-5-methyl-2,4-piperidinedione (methyprylon), metopon, 8-chloro-6-(2-fluorophenyl)-1-methyl-4H-imidazo[1,5-a][1,4]benzodiazepine (midazolam), 2-(benzhydrylsulfinyl)acetamide (modafinil), 4,5α-epoxy-17-methyl-7-morphinen-3,6α-diol (morphine), myrophine, (±)-trans-3-(1,1-dimethylheptyl)-7,8,10,10α-tetrahydro-1-hydroxy-6,6-dimethyl-6H-dibenzo-[b,d]pyran-9(6αH)-one (nabilone), nalbuphene, nalorphine, narceine, nicomorphine, 1-methyl-7-nitro-5-phenyl-1H-1,4-benzodiazepin-2(3H)-one (nimetazepam), 7-nitro-5-phenyl-1H-1,4-benzodiazepin-2(3H)-one (nitrazepam), 7-chloro-5-phenyl-1H-1,4-benzodiazepin-2(3H)-one (nordazepam), norlevorphanol, 6-dimethylamino-4,4-diphenyl-3-hexanone (normethadone), normorphine, norpipanone, the exudation from plants belonging to the species Papaver somniferum (opium), 7-chloro-3-hydroxy-5-phenyl-1H-1,4-benzodiazepin-2(3H)-one (oxazepam), (cis-trans)-10-chloro-2,3,7,11b-tetrahydro-2-methyl-11b-phenyloxazolo[3,2-d][1,4]benzodiazepin-6-(5H)-one (oxazolam), 4,5α-epoxy-14-hydroxy-3-methoxy-17-methyl-6-morphinanone (oxycodone), oxymorphone, plants and parts of plants belonging to the species Papaver somniferum (including the subspecies setigerum) (Papaver somniferum), papaveretum, 2-imino-5-phenyl-4-oxazolidinone (pemoline), 1,2,3,4,5,6-hexahydro-6,11-dimethyl-3-(3-methyl-2-butenyl)-2,6-methano-3-benzazocin-8-ol (pentazocine), 5-ethyl-5-(1-methylbutyl)-barbituric acid (pentobarbital), ethyl (1-methyl-4-phenyl-4-piperidinecarboxylate) (pethidine), phenadoxone, phenomorphane, phenazocine, phenoperidine, piminodine, pholcodeine, 3-methyl-2-phenylmorpholine (phenmetrazine), 5-ethyl-5-phenylbarbituric acid (phenobarbital), α,α-dimethylphenethylamine (phentermine), 7-chloro-5-phenyl-1-(2-propynyl)-1H-1,4-benzodiazepin-2(3H)-one (pinazepam), α-(2-piperidyl)benzhydryl alcohol (pipradrol), 1′-(3-cyano-3,3-diphenylpropyl)[1,4′-bipiperidine]-4′-carboxamide (piritramide), 7-chloro-1-(cyclopropylmethyl)-5-phenyl-1H-1,4-benzodiazepin-2(3H)-one (prazepam), premethadone, profadol, proheptazine, promedol, properidine, propoxyphene, N-(1-methyl-2-piperidinoethyl)-N-(2-pyridyl)propionamide, methyl {3-[4-methoxycarbonyl-4-(N-phenylpropanamido)piperidino]propanoate} (remifentanil), 5-sec-butyl-5-ethylbarbituric acid (secbutabarbital), 5-allyl-5-(1-methylbutyl)-barbituric acid (secobarbital), N-{4-methoxymethyl-1-[2-(2-thienyl)ethyl]-4-piperidyl}propionanilide (sufentanil), 7-chloro-2-hydroxy-methyl-5-phenyl-1H-1,4-benzodiazepin-2(3H)-one (temazepam), 7-chloro-5-(1-cyclohexenyl)-1-methyl-1H-1,4-benzodiazepin-2(3H)-one (tetrazepam), ethyl (2-dimethylamino-1-phenyl-3-cyclohexene-1-carboxylate) (tilidine (cis and trans)), tramadol, 8-chloro-6-(2-chlorophenyl)-1-methyl-4H-[1,2,4]triazolo[4,3-a][1,4]benzodiazepine (triazolam), 5-(1-methylbutyI)-5-vinylbarbituric acid (vinylbital), (1R*,2R*)-3-(3-dimethylamino-1-ethyl-2-methyl-propyl)phenol, (1R,2R,4S)-2-(dimethylamino)methyl-4-(p-fluorobenzyloxy)-1-(m-methoxyphenyl)cyclohexanol, (1R,2R)-3-(2-dimethylaminomethyl-cyclohexyl)phenol, (1S,2S)-3-(3-dimethylamino-1-ethyl-2-methyl-propyl)phenol, (2R,3R)-1-dimethylamino-3(3-methoxyphenyl)-2-methyl-pentan-3-ol, (1RS,3RS,6RS)-6-dimethylaminomethyl-1-(3-methoxyphenyl)-cyclohexane-1,3-diol, 3-(2-dimethylaminomethyl-1-hydroxy-cyclohexyl)phenyl 2-(4-isobutoxy-phenyl)propionate, 3-(2-dimethylaminomethyl-1-hydroxy-cyclohexyl)phenyl 2-(6-methoxy-naphthalen-2-yl)propionate, 3-(2-dimethylaminomethyl-cyclohex-1-enyl)-phenyl 2-(4-isobutyl-phenyl)propionate, 3-(2-dimethylaminomethyl-cyclohex-1-enyl)-phenyl 2-(6-methoxy-naphthalen-2-yl)propionate, (RR-SS)-2-acetoxy-4-trifluoromethyl-benzoic acid 3-(2-dimethylaminomethyl-1-hydroxy-cyclohexyl)-phenyl ester, (RR-SS)-2-hydroxy-4-trifluoromethyl-benzoic acid 3-(2-dimethylaminomethyl-1-hydroxy-cyclohexyl)-phenyl ester, (RR-SS)-4-chloro-2-hydroxy-benzoic acid 3-(2-dimethylaminomethyl-1-hydroxy-cyclohexyl)-phenyl ester, (RR-SS)-2-hydroxy-4-methyl-benzoic acid 3-(2-dimethylaminomethyl-1-hydroxy-cyclohexyl)-phenyl ester, (RR-SS)-2-hydroxy-4-methoxy-benzoic acid 3-(2-dimethylaminomethyl-1-hydroxy-cyclohexyl)-phenyl ester, (RR-SS)-2-hydroxy-5-nitro-benzoic acid 3-(2-dimethylaminomethyl-1-hydroxy-cyclohexyl)-phenyl ester, (RR-SS)-2′,4′-difluoro-3-hydroxy-biphenyl-4-carboxylic acid 3-(2-dimethylaminomethyl-1-hydroxy-cyclohexyl)-phenyl ester together with corresponding stereoisomeric compounds, in each case the corresponding derivatives thereof, in particular amides, esters or ethers, and in each case the physiologically acceptable compounds thereof, in particular the salts and solvates thereof.

The compounds (1R*,2R*)-3-(3-dimethylamino-1-ethyl-2-methyl-propyl)phenol, (1 R,2R,4S)-2-(dimethylamino)methyl-4-(p-fluorobenzyloxy)-1-(m-methoxyphenyl)cyclohexanol or the stereoisomeric compounds thereof or the physiologically acceptable compounds thereof, in particular the hydrochlorides thereof, the derivatives thereof, such as esters or ethers, and processes for the production thereof are known, for example, from EP-A-693475 or EP-A-780369. The corresponding descriptions are hereby introduced as a reference and are deemed to be part of the disclosure.

Active ingredients which may particularly preferably be protected against abuse according to the invention are oxycodone, morphine, hydromorphone, tramadol or the physiologically acceptable salts thereof.

By using ultrasound in combination with the binder in the process according to the invention, it is possible simply and reproducibly to achieve the necessary breaking strength which is required considerably to complicate or to prevent pulverisation of the dosage form with conventional means and hence any subsequent abuse.

Using the process according to the invention, it is possible to obtain dosage forms in the form of tablets, microtablets, suppositories, granules, microparticles, spheroids or pellets. The multiparticulate forms preferably have a size or size distribution in the range from 0.1 to 3 mm, particularly preferably in the range from 0.5 to 2 mm.

Oral dosage forms are preferably produced using the process according to the invention.

The process according to the invention is performed by initially producing a homogeneous mixture of at least one active ingredient with potential for abuse and at least one binder. Further auxiliary substances, such as for example fillers, plasticisers, slip agents or dyes, may also be incorporated into this mixture. A low molecular weight polyethylene glycol is preferably used as plasticiser.

Mixing may be performed with the assistance of conventional mixers. Examples of suitable mixers are roll mixers, which are also known as tumbler, drum or rotary mixers, container mixers, barrel mixers (drum hoop mixers or tumbling mixers) or shaking mixers, shear mixers, compulsory mixers, plough bar mixers, planetary kneader-mixers, Z kneaders, sigma kneaders, fluid mixers or high-intensity mixers.

Selection of the suitable mixer is determined inter alia by the flowability and cohesiveness of the material to be mixed.

The mixture is then subjected to shaping. The mixture is preferably shaped during or after ultrasonication, preferably by compaction.

### BRIEF DESCRIPTION OF THE DRAWING

The invention will now be described in greater detail with reference to the drawing, wherein:

FIG. 1 shows an ultrasound device useful in carrying out the present invention.

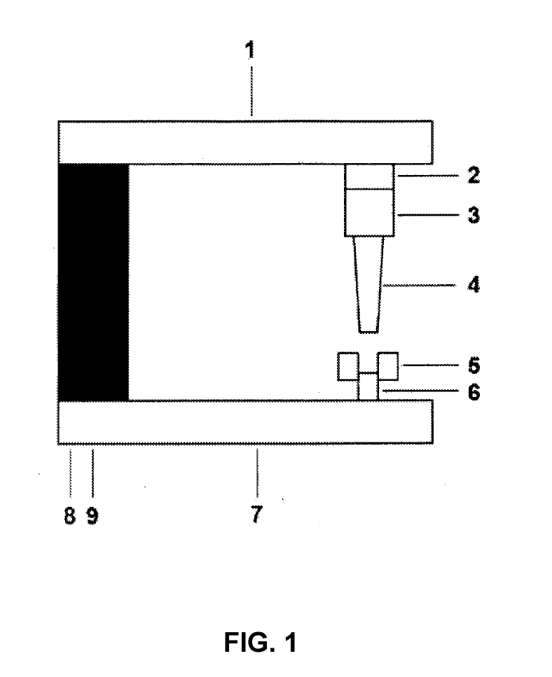

It is particularly preferred during ultrasonication that there is direct contact between the mixture and the sonotrode of the ultrasound device. An ultrasound device as shown in FIG. 1 is preferably used in the process according to the invention.

In this FIG. 1, (1) denotes the press, with which the necessary force is applied, (2) the converter, (3) the booster, (4) the sonotrode, (5) the shaping die, (6) the bottom punch, (7) the base plate, (8) and (9) the ultrasound generator and device controller.

A frequency of 1 kHz to 2 MHz, preferably of 15 to 40 kHz, should be maintained during ultrasonication. Ultrasonication should be performed until softening of the binder is achieved. This is preferably achieved within a few seconds, particularly preferably within 0.1 to 5 seconds, preferably 0.5 to 3 seconds.

Ultrasonication and the application of force ensure uniform energy transfer, so bringing about rapid and homogeneous sintering of the mixture. In this manner, dosage forms are obtained which have a breaking strength of ≧500 N and thus cannot be pulverised.

Before shaping is performed, the mixture may be pelletised after the mixing operation, after which the resultant granules are shaped into the dosage form, such as tablets, with ultrasonication and application of force.

Pelletisation may be performed in machinery and apparatus known to the person skilled in the art.

If pelletisation is performed as wet pelletisation, water or aqueous solutions, such as for example ethanol/water or isopropanol/water, may be used as the pelletisation liquid.

The mixture or the granules produced therefrom may also be subjected to melt extrusion for further shaping, wherein the mixture is converted into a melt by ultrasonication and exposure to force and then extruded through a dies. The strands or strand obtained in this manner may be singulated to the desired length using known apparatus. The formed articles singulated in this manner may optionally furthermore be converted into the final shape with ultrasonication and application of force.

Final shaping to yield the dosage form preferably proceeds with application of force in appropriate moulds.

The above-described formed articles may also be produced with a calendering process by initially plasticising the mixture or the granules produced therefrom by means of ultrasonication and application of force and performing extrusion through an appropriate die. These extrudates are then shaped into the final shape between two contrarotating shaping rolls, preferably with application of force.

As already mentioned, shaping to yield the final shape of the dosage form preferably proceeds by using a mixture comprising the active ingredient with potential for abuse and the binder with a breaking strength of ≧500 N in powder form by direct compression with application of force, wherein this mixture is ultrasonicated before or during application of force. The force is at most the force which is conventionally used for shaping dosage forms, such as tablets, or for press-moulding granules into the corresponding final shape.

The tablets produced according to the invention may also be multilayer tablets.

In the case of multilayer tablets, at least the active ingredient layer must be subjected ultrasonication and application of force.

The corresponding necessary application of force may also be applied to the mixture with the assistance of extruder rolls or calender rolls. Shaping of the dosage form preferably proceeds by direct press-moulding of a pulverulent mixture of the components of the dosage form or corresponding granules formed therefrom, wherein ultrasonication preferably proceeds during or before shaping. This ultrasonication proceeds until the binder has softened, which is conventionally achieved in less than 1 second to at most 5 seconds.

In order to achieve the necessary breaking strength, at least one binder with a breaking strength of ≧500 N is used in the production process according to the invention. The binder is preferably used in a quantity of at least 20 wt. %, preferably of at least 35 wt. %, particularly preferably of 50 to 99.9 wt. %, relative to the mixture of active ingredient and binder. The binder used for this purpose is at least one polymer selected from among the group comprising polymethylene oxide, polyethylene oxide, polypropylene oxide, polyethylene, polypropylene, polyvinyl chloride, polycarbonate, polystyrene, polyacrylate, poly(hydroxyfatty acids), such as for example poly(3-hydroxybutyrate-co-3-hydroxyvalerate) (Biopol®), poly(hydroxyvaleric acid), polycaproplactone, polyvinyl alcohol, polyesteramides, polyethylene succinate, polylactones, polyglycolides, polyurethanes, polyamides, polylactides, polylactide/glycolide, polylactones, polyglycolides, polyorthoesters, polyanhydrides, block polymers of polyethylene glycol and polybutylene terephthalate (Polyactive®), polyanhydrides (Polifeprosan), the copolymers thereof, and mixtures of at least two of the stated polymers. The polymers are distinguished by a molecular weight of at least 0.5 million, determined by rheological measurements. Thermoplastic polyalkylene oxides, such as polyethylene oxides, with a molecular weight of at least 0.5 million, preferably of at least 5 million, preferably of up to 15 million, determined by rheological measurements, are very particularly preferred. These polymers have a viscosity at 25° C. of 4500 to 17600 cP, measured on a 5 wt. % aqueous solution using a model RVF Brookfield viscosimeter (spindle no. 2/rotational speed 2 rpm), of 400 to 4000 cP, measured on a 2 wt. % aqueous solution using the stated viscosimeter (spindle no. 1 or 3/rotational speed 10 rpm) or of 1650 to 10000 cP, measured on a 1 wt. % aqueous solution using the stated viscosimeter (spindle no. 2/rotational speed 2 rpm).

The polymers are preferably used in powder form.

Thanks to the use of binder and the ultrasonication with application of force, it is possible to obtain dosage forms with a breaking strength of ≧500 N.

In order to achieve the necessary breaking strength with the production process according to the invention, it is furthermore possible additionally to use at least one natural or synthetic wax with a breaking strength, measured using the method disclosed in the present application, of at least 500 N. Waxes with a softening point of at least 60° C. are preferred. Carnauba wax and beeswax are particularly preferred. Carnauba wax is very particularly preferred. Carnauba wax is a natural wax which is obtained from the leaves of the carnauba palm and has a softening point of ≧80° C. When the wax component is additionally used, it is used together with at least one polymer in quantities such that the dosage form has a breaking strength of at least 500 N.

The dosage forms obtained by the production process according to the invention are distinguished in that, due to their hardness, they cannot be pulverised, for example by grinding in a mortar. This virtually rules out oral or parenteral, in particular intravenous or nasal abuse. However, in order to prevent any possible abuse of the dosage forms obtained by the production process according to the invention in the event of comminution and/or pulverisation which possibly occur nonetheless due to extraordinary force, in a preferred embodiment these dosage forms may contain further abuse-complicating or -preventing agents as auxiliary substances.

The dosage forms obtained by the production process according to the invention may accordingly additionally comprise, apart from one or more active ingredients with potential for abuse and a binder, at least one of the following components:
(a) at least one substance which irritates the nasal passages and/or pharynx,
(b) at least one viscosity-increasing agent, which, with the assistance of a necessary minimum quantity of an aqueous liquid, forms a gel with the extract obtained from the dosage form, which gel preferably remains visually distinguishable when introduced into a further quantity of an aqueous liquid,
(c) at least one antagonist for each of the active ingredients with potential for abuse,
(d) at least one emetic,
(e) at least one dye as an aversive agent,
(f) at least one bitter substance.

Components (a) to (f) are additionally each individually suitable for abuse-proofing the dosage forms obtained by the production process according to the invention. Accordingly, component (a) is preferably suitable for proofing the dosage form against nasal, oral and/or parenteral, preferably intravenous, abuse, component (b) is preferably suitable for proofing against parenteral, particularly preferably intravenous and/or nasal abuse, component (c) is preferably suitable for proofing against nasal and/or parenteral, particularly preferably intravenous, abuse, component (d) is preferably suitable for proofing against parenteral, particularly preferably intravenous, and/or oral and/or nasal abuse, component (e) is suitable as a visual deterrent against oral or parenteral abuse and component (f) is suitable for proofing against oral or nasal abuse. Combined use according to the invention of at least one of the above-stated components makes it possible still more effectively to prevent abuse of dosage forms obtained by the production process according to the invention.

For example, the dosage form obtained by the process according to the invention may also comprise two or more of components (a)-(f) in a combination, preferably (a), (b) and optionally (c) and/or (f) and/or (e) or (a), (b) and optionally (d) and/or (f) and/or (e).

In another embodiment, the dosage form according to the invention may comprise all of components (a)-(f).

If the dosage form obtained by the process according to the invention comprises an abuse-preventing component (a), substances which irritate the nasal passages and/or pharynx which may be considered according to the invention are any substances which, when administered abusively via the nasal passages and/or pharynx, bring about a physical reaction which is either so unpleasant for the abuser that he/she does not wish to or cannot continue administration, for example burning, or physiologically counteracts taking of the corresponding active ingredient, for example due to increased nasal secretion or sneezing. These substances which conventionally irritate the nasal passages and/or pharynx may also bring about a very unpleasant sensation or even unbearable pain when administered parenterally, in particular intravenously, such that the abuser does not wish to or cannot continue taking the substance.

Particularly suitable substances which irritate the nasal passages and/or pharynx are those which cause burning, itching, an urge to sneeze, increased formation of secretions or a combination of at least two of these stimuli. Appropriate substances and the quantities thereof which are conventionally to be used are known per se to the person skilled in the art or may be identified by simple preliminary testing.

The substance which irritates the nasal passages and/or pharynx of component (a) is preferably based on one or more constituents or one or more plant parts of at least one hot substance drug.

Corresponding hot substance drugs are known per se to the person skilled in the art and are described, for example, in “Pharmazeutische Biologie—Drogen and ihre Inhaltsstoffe” by Prof. Dr. Hildebert Wagner, 2nd., revised edition, Gustav Fischer Verlag, Stuttgart-New York, 1982, pages 82 et seq. The corresponding description is hereby introduced as a reference and is deemed to be part of the disclosure.

The dosage form obtained by the process according to the invention may preferably contain the plant parts of the corresponding hot substance drugs in a quantity of 0.01 to 30 wt. %, particularly preferably of 0.1 to 0.5 wt. %, in each case relative to the total weight of the dosage unit.

If one or more constituents of corresponding hot substance drugs are used, the quantity thereof in a dosage unit obtained by the process according to the invention preferably amounts to 0.001 to 0.005 wt. %, relative to the total weight of the dosage unit.

A dosage unit is taken to mean a separate or separable administration unit, such as for example a tablet or a capsule.

One or more constituents of at least one hot substance drug selected from the group comprising Allii sativi bulbus (garlic), Asari rhizoma cum herba (Asarum root and leaves), Calami rhizoma (calamus root), Capsici fructus (capsicum), Capsici fructus acer (cayenne pepper), Curcumae longae rhizoma (turmeric root), Curcumae xanthorrhizae rhizoma (Javanese turmeric root), Galangae rhizoma (galangal root), Myristicae semen (nutmeg), Piperis nigri fructus (pepper), Sinapis albae semen (white mustard seed), Sinapis nigri semen (black mustard seed), Zedoariae rhizoma (zedoary root) and Zingiberis rhizoma (ginger root), particularly preferably from the group comprising Capsici fructus (capsicum), Capsici fructus acer (cayenne pepper) and Piperis nigri fructus (pepper) may preferably be added as component (a) to the dosage form obtained by the process according to the invention.

The constituents of the hot substance drugs preferably comprise o-methoxy(methyl)phenol compounds, acid amide compounds, mustard oils or sulfide compounds or compounds derived therefrom.

Particularly preferably, at least one constituent of the hot substance drugs is selected from the group consisting of myristicin, elemicin, isoeugenol, α-asarone, safrole, gingerols, xanthorrhizol, capsaicinoids, preferably capsaicin, capsaicin derivatives, such as N-vanillyl-9E-octadecenamide, dihydrocapsaicin, nordihydrocapsaicin, homocapsaicin, norcapsaicin and nomorcapsaicin, piperine, preferably trans-piperine, glucosinolates, preferably based on non-volatile mustard oils, particularly preferably based on p-hydroxybenzyl mustard oil, methylmercapto mustard oil or methylsulfonyl mustard oil, and compounds derived from these constituents.

Another option for preventing abuse of the dosage form obtained by the process according to the invention consists in adding at least one viscosity-increasing agent as a further abuse-preventing component (b) to the dosage form, which, with the assistance of a necessary minimum quantity of an aqueous liquid, forms a gel with the extract obtained from the dosage form, which gel is virtually impossible to administer safely and preferably remains visually distinguishable when introduced into a further quantity of an aqueous liquid.

For the purposes of the present invention, visually distinguishable means that the active ingredient-containing gel formed with the assistance of a necessary minimum quantity of aqueous liquid, when introduced, preferably with the assistance of a hypodermic needle, into a further quantity of aqueous liquid at 37° C., remains substantially insoluble and cohesive and cannot straightforwardly be dispersed in such a manner that it can safely be administered parenterally, in particular intravenously. The material preferably remains visually distinguishable for at least one minute, preferably for at least 10 minutes.

The increased viscosity of the extract makes it more difficult or even impossible for it to be passed through a needle or injected. If the gel remains visually distinguishable, this means that the gel obtained on introduction into a further quantity of aqueous liquid, for example by injection into blood, initially remains in the form of a largely cohesive thread, which, while it may indeed be broken up mechanically into smaller fragments, cannot be dispersed or even dissolved in such a manner that it can safely be administered parenterally, in particular intravenously. In combination with at least one optionally present component (a) or c to (e), this additionally leads to unpleasant burning, vomiting, bad flavour and/or visual deterrence.

Intravenous administration of such a gel would therefore most probably result in serious damage to the health of the abuser.

In order to verify whether a viscosity-increasing agent is suitable as component (b) for use in the dosage form obtained by the production process according to the invention, the active ingredient is mixed with the viscosity-increasing agent and suspended in 10 ml of water at a temperature of 25° C. If this results in the formation of a gel which fulfils the above-stated conditions, the corresponding viscosity-increasing agent is suitable for preventing or averting abuse of the dosage forms obtained by the process according to the invention.

If component (b) is added to the dosage form obtained by the process according to the invention, preferably one or more viscosity-increasing agents are used, which are selected from the group comprising microcrystalline cellulose with 11 wt. % carboxymethylcellulose sodium (Avicel® RC 591), carboxymethylcellulose sodium (Blanose®, CMC-Na C300P®, Frimulsion BLC-5®, Tylose C300 P®), polyacrylic acid (Carbopol® 980 NF, Carbopol® 981), locust bean flour (Cesagum® LA-200, Cesagum® LID/150, Cesagum® LN-1), pectins, preferably from citrus fruits or apples (Cesapectin® HM Medium Rapid Set), waxy maize starch (C*Gel 04201®), sodium alginate (Frimulsion ALG (E401)®), guar flour (Frimulsion BM®, Polygum 26/1-75®), iota-carrageenan (Frimulsion D021®), karaya gum, gellan gum (Kelcogel F®, Kelcogel LT100®), galactomannan (Meyprogat 150®), tara stone flour (Polygum 43/1®), propylene glycol alginate (Protanal-Ester SD-LB®), sodium hyaluronate, tragacanth, tara gum (Vidogum SP 200®), fermented polysaccharide welan gum (K1A96), xanthans such as xanthan gum (Xantural 180®). Xanthans are particularly preferred. The names stated in brackets are the trade names by which the materials are known commercially. In general, a quantity of 0.1 to 5 wt. % of the viscosity-increasing agent(s) is sufficient to fulfil the above-stated conditions.

The component (b) viscosity-increasing agents, where provided, are preferably present in the dosage form obtained by the production process according to the invention in quantities of 0.1 to 25 wt. %, preferably of 0.5 to 15 wt. %, particularly preferably of 1-10 wt. %, per dosage unit, i.e. per administration unit.

In a particularly preferred embodiment of the present invention, the viscosity-increasing agents used as component (b) are those which, on extraction from the dosage form with the necessary minimum quantity of aqueous liquid, form a gel which encloses air bubbles. The resultant gels are distinguished by a turbid appearance, which provides the potential abuser with an additional optical warning and discourages him/her from administering the gel parenterally.

It is also possible to formulate the viscosity-increasing agent and the other constituents in the dosage form obtained by the production process according to the invention in a mutually spatially separated arrangement.

In order to discourage and prevent abuse, the dosage form obtained by the process according to the invention may furthermore comprise component (c), namely one or more antagonists for the active ingredient or active ingredients with potential for abuse, wherein the antagonists are preferably spatially separated from the remaining constituents of the dosage form obtained by the process according to the invention and, when correctly used, do not exert any effect.

Suitable antagonists for preventing abuse of the active ingredients are known per se to the person skilled in the art and may be present in the dosage form obtained by the production process according to the invention as such or in the form of corresponding derivatives, in particular esters or ethers, or in each case in the form of corresponding physiologically acceptable compounds, in particular in the form of the salts or solvates thereof.

If the active ingredient present in the dosage form is an opiate or an opioid, the antagonist used is preferably an antagonist selected from the group comprising naloxone, naltrexone, nalmefene, nalide, nalmexone, nalorphine or naluphine, in each case optionally in the form of a corresponding physiologically acceptable compound, in particular in the form of a base, a salt or solvate. The corresponding antagonists, where component (c) is provided, are preferably used in a quantity of ≧10 mg, particularly preferably in a quantity of 10 to 100 mg, very particularly preferably in a quantity of 10 to 50 mg per dosage form, i.e. per administration unit.

If the dosage form obtained by the process according to the invention comprises a stimulant as active ingredient, the antagonist is preferably a neuroleptic, preferably at least one compound selected from the group comprising haloperidol, promethazine, fluphenazine, perphenazine, levomepromazine, thioridazine, perazine, chlorpromazine, chlorprothixine, zuclopentixol, flupentixol, prothipendyl, zotepine, benperidol, pipamperone, melperone and bromperidol.

The dosage form obtained by the process according to the invention preferably comprises these antagonists in a conventional therapeutic dose known to the person skilled in the art, particularly preferably in a quantity of twice to three times the conventional dose per administration unit.

If the combination for discouragement and prevention of abuse of the dosage form obtained by the process according to the invention comprises component (d), it may comprise at least one emetic, which is preferably present in a spatially separated arrangement from the other components of the dosage form obtained by the process according to the invention and, when correctly used, is intended not to exert its effect in the body.

Suitable emetics for preventing abuse of an active ingredient are known to the person skilled in the art and may be present in the dosage form obtained by the process according to the invention as such or in the form of corresponding derivatives, in particular esters or ethers, or in each case in the form of corresponding physiologically acceptable compounds, in particular in the form of the salts or solvates thereof.

An emetic based on one or more constituents of ipecacuanha (ipecac) root, preferably based on the constituent emetine may preferably be considered in the dosage form obtained by the process according to the invention, as are, for example, described in “Pharmazeutische Biologie—Drogen and ihre Inhaltsstoffe” by Prof. Dr. Hildebert Wagner, 2nd, revised edition, Gustav Fischer Verlag, Stuttgart, New York, 1982. The corresponding literature description is hereby introduced as a reference and is deemed to be part of the disclosure.

The dosage form obtained by the process according to the invention may preferably comprise the emetic emetine as component (d), preferably in a quantity of ≧10 mg, particularly preferably of ≧20 mg and very particularly preferably in a quantity of ≧40 mg per dosage form, i.e. administration unit.

Apomorphine may likewise preferably be used as an emetic for additional abuse-proofing, preferably in a quantity of preferably ≧3 mg, particularly preferably of ≧5 mg and very particularly preferably of ≧7 mg per administration unit.

If the dosage form obtained by the process according to the invention contains component (e) as an additional abuse-preventing auxiliary substance, the use of such a dye brings about an intense coloration of a corresponding aqueous solution, in particular when the attempt is made to extract the active ingredient for parenteral, preferably intravenous administration, which coloration may act as a deterrent to the potential abuser. Oral abuse, which conventionally begins by means of aqueous extraction of the active ingredient, may also be prevented by this coloration. Suitable dyes and the quantities required for the necessary deterrence may be found in WO 03/015531, wherein the corresponding disclosure should be deemed to be part of the present disclosure and is hereby introduced as a reference.

If the dosage form obtained by the process according to the invention contains component (f) as a further abuse-preventing auxiliary substance, this addition of at least one bitter substance and the consequent impairment of the flavour of the dosage form additionally prevents oral and/or nasal abuse.

Suitable bitter substances and the quantities effective for use may be found in US-2003/0064099 A1, the corresponding disclosure of which should be deemed to be the disclosure of the present application and is hereby introduced as a reference. Suitable bitter substances are preferably aromatic oils, preferably peppermint oil, eucalyptus oil, bitter almond oil, menthol, fruit aroma substances, preferably aroma substances from lemons, oranges, limes, grapefruit or mixtures thereof, and/or denatonium benzoate.

Method for Determining Breaking Strength

In order to verify whether a polymer may be used as binder with a breaking strength of ≧500 N, the polymer is press-moulded to form a tablet with a diameter of 10 mm and a height of 5 mm using a force of 150 N at a temperature which at least corresponds to the softening point of the polymer (determined with the assistance of a DSC diagram of the polymer). Using tablets produced in this manner, breaking strength is determined with the apparatus described below in accordance with the method for determining the breaking strength of tablets published in the European Pharmacopoeia 1997, page 143, 144, method no. 2.9.8. The apparatus used for the measurement is a “Zwick Z 2.5” materials tester, Fmax=2.5 kN, draw max. 1150 mm with the setup comprising 1 column and 1 spindle, clearance behind of 100 mm, a test speed of 0.1800 mm/min and testControl software. Measurement was performed using a pressure piston with screw-in inserts and a cylinder (diam. 10 mm), a force transducer, Fmax. 1 kN, diameter=8 mm, class 0.5 from 10 N, class 1 from 2 N to ISO 7500-1, with manufacturers test certificate M to DIN 55350-18 (Zwick gross force Fmax=1.45 kN) (all apparatus from Zwick GmbH & Co. KG, Ulm, Germany).

In order to verify whether the polymer may be plasticised by means of ultrasound, it is treated by means of a force of 500 N and ultrasound. If the polymer is plasticised, it is in principle suitable for the process according to the invention.

The tablets deemed to be resistant to breaking under a specific load include not only those which have not broken but also those which may have suffered plastic deformation under the action of the force.

The breaking strength of a dosage form obtained according to the invention, provided it is in the form of a tablet or pellet, may be determined using the same measurement method.

The invention is explained below with reference to Examples. These explanations are given merely by way of example and do not restrict the general concept of the invention.

### EXAMPLES

### Example 1

| Components | Per tablet | Complete batch |
|---|---|---|
| Tramadol HCl | 205.0 mg | 6.13 g |
| Polyethylene oxide, NF, MFI | 381.0 mg | 11.38 g |
| (190° C. at 21.6 kg/10 min) MW 7 000 000 | | |
| (Polyox WSR 303, Dow Chemicals) | | |
| Total weight | 586.0 mg | 17.51 g |

Tramadol hydrochloride and polyethylene oxide powder were mixed in a free-fall mixer. The mixture was then pressed into tablets with ultrasonication and application of the force stated below. The following machine was used for this purpose:

Press: Branson WPS, 94-003-A, pneumatic (Branson Ultraschall, Dietzenbach, Germany)

Generator (2000 W): Branson PG-220A, 94-001-A analogue (Branson Ultraschall)

The diameter of the sonotrode was 12 mm. The press surface was flat.

The following parameters were selected for plasticisation of the mixture:

Frequency: 20 Hz

Amplitude: 50%

Force: 250 N

Ultrasonication and application of force: 0.5 seconds

The breaking strength of the tablets is determined with the stated apparatus in accordance with the stated method. No breakage occurred when a force of 500 N was applied. The tablet could not be comminuted using a hammer, nor with the assistance of a pestle and mortar.

In vitro release of the active ingredient from the preparation was determined in a paddle stirrer apparatus with sinker in accordance with Pharm. Eur. The temperature of the release medium was 37° C. and the rotational speed of the stirrer 75 min⁻¹. The release medium used was intestinal juice, pH 6.8. The quantity of active ingredient released in each case into the medium at any one time was determined by spectrophotometry.

| | Quantity of active ingredient released |
|---|---|
| Time | Tramadol |
| 30 min | 13% |
| 240 min | 51% |
| 480 min | 76% |
| 720 min | 100% |

What is claimed:

1. A process for the production of an abuse-proofed solid dosage form comprising at least one active ingredient with potential for abuse and at least one binder, the at least one active ingredient with potential for abuse being selected from the group consisting of oxymorphone and physiologically acceptable compounds and derivatives thereof, said dosage form having a breaking strength of at least 500 N, and said process comprising exposing a mixture comprising the at least one active ingredient and the at least one binder to ultrasound and force.

2. A process according to claim 1, wherein the dosage form is an oral dosage form.

3. A process according to claim 1, wherein the physiologically acceptable compounds and derivatives are selected from the group consisting of salts, solvates, esters, ethers and amides.

4. A process according to claim 1, wherein the active ingredient with potential for abuse is selected from the group consisting of morphine, hydromorphone, and the physiologically acceptable salts thereof.

5. A process according to claim 1, wherein the binder is present in a quantity of at least 20 wt. % relative to the total weight of the dosage form.

6. A process according to claim 1, wherein the binder is at least one synthetic or natural polymer and optionally a wax.

7. A process according to claim 6, wherein the polymer exhibits a viscosity at 25° C. of 4500 to 17600 cP, measured on a 5 wt. % aqueous solution with the assistance of a Brookfield viscosimeter.

8. A process according to claim 7, wherein the polymer is at least one polymer selected from among the group consisting of polyethylene oxides, polyethylenes, polypropylenes, polyvinyl chlorides, polycarbonates, polystyrenes, polyacrylates and the copolymers thereof.

9. A process according to claim 8, wherein the polymer is a polyethylene oxide and the polyethylene oxide has a molecular weight of at least 1 million g/mol.

10. A process according to claim 1, wherein, apart from the active ingredient with potential for abuse and the binder, the dosage form also comprises at least one further auxiliary substance.

11. A process according to claim 10, wherein the at least one further auxiliary substance is a plasticiser.

12. A process according to claim 1, wherein the ultrasound has a frequency of 1 kHz to 2 MHz.

13. A process according to claim 1, wherein the mixture directly contacts the ultrasound source during ultrasonication.

14. A process according to claim 1, wherein ultrasonication proceeds until the binder has softened.

15. A process according to claim 1, which further comprises shaping the mixture by compaction during or after ultrasonication or by extrusion with rollers and/or by calendering during or after ultrasonication.

16. A process according to claim 15, which further comprises applying a force for the purpose of compaction.

17. A process according to claim 15, which further comprises compaction, wherein the mixture is in the form of powder, pellets, microparticles or granules.

18. A process according to claim 1, which further comprises shaping the mixture into tablets.

19. A process according to claim 1, which further comprises shaping the mixture into a multiparticulate final shape.
